(19) United States
(12) Patent Application Publication
(10) Pub. No.: US 2024/0001366 A1
(43) Pub. Date: Jan. 4, 2024

(54) SAMPLE COLLECTION DEVICE AND METHODS OF USE

(71) Applicants: QUANTIGEN, LLC, Fishers, IN (US); PIONEER HI-BRED INTERNATIONAL, INC., Johnston, IA (US)

(72) Inventors: Martin BAKSTAD, Jelcz-Laskowice (PL); James ELLIOTT, Fishers, IN (US); Simon EXELL, Jelcz-Laskowice (PL); Rafal GRABICKI, Jelcz-Laskowice (PL); Leah PADGETT, Fishers, IN (US); James RAINS, Fishers, IN (US); Christopher REPPE, Johnston, IA (US); Hillary SULLIVAN, Johnston, IA (US)

(21) Appl. No.: 18/271,107

(22) PCT Filed: Jan. 6, 2022
(86) PCT No.: PCT/US2022/011410

§ 371 Date: Jul. 6, 2023

Related U.S. Application Data
(60) Provisional application No. 63/134,903, filed on Jan. 7, 2021.

(51) Int. Cl. B01L 3/00

(52) U.S. Cl. CPC B01L 3/5029; B01L 2300/042; B01L 2300/047

(57) ABSTRACT

The present disclosure provides a device for collecting a biological sample. The device may include a tube, a primary cap which engages an open end of the tube, and a secondary cap which engages the primary cap. The device may include a collection member, which may include a swab. Methods of collecting and analyzing samples are also disclosed.

### RELATED APPLICATIONS

This application claims the benefit of U.S. Provisional Application No. 63/134,903, filed Jan. 7, 2021, which is incorporated by reference herein in its entirety.

### BACKGROUND

The present disclosure relates to devices, systems, and methods useful for collecting and analyzing biological specimens.

Large-scale collection of biological samples for high-throughput screening presents a number of challenges. The global coronavirus pandemic has provided a stark reminder of the structural barriers which hamper high-throughput screening for incectious pathogens specifically. In this context, sample collection generally occurs in an in-clinic setting, requiring physical proximity between skilled personnel and patients in a confined space, using medical facilities that could be dedicated to other needs, and risking transmission of virus. Further, test samples can be collected at a first location and analyzed at a second, which in some cases necessitates the consideration of thermal stability of samples during shipment.

One challenge specific to high-throughput screening is the variability in collection devices, tubes, and shipping container sizes used by different laboratories and manufactured by different companies. This variety increases in-lab sample management and automation complexity as different containers and sampling devices may need to be processed in different ways. Additionally, many clinical samples are analyzed by interrogating the sample for nucleic acid (DNA or RNA) content. Some sample matrices inhibit or prevent downstream reactions that provide a reliable report from the assay, making nucleic acid extraction an indispensible part of the workflow. Extraction systems effectively double the instrumentation required for testing.

### BRIEF SUMMARY

In one aspect, the present disclosure provides a device for collecting a biological sample. The device may include a tube defining a tube body and extending from an open end to a closed end. The tube body may define an exterior surface, an interior surface, and a tube volume bounded by the interior surface. The closed end may include at least one projection on a portion of the exterior surface, the projection being capable of engagement with a component of an analytical apparatus. The device may include a collection assembly engageable with the open end of the tube. The collection assembly may include a primary cap defining a cap body and extending from a first end to a second end, the cap body defining an outer surface. The cap body may include a first portion including the first end and defining a receiving portion, and a second portion including the second end, the second portion defining a cavity open to the second end. The collection assembly may include a secondary cap which includes a sealing portion, the sealing portion sized to fit in the cavity of the primary cap. In one aspect, the present disclosure provides for a system including this device and an analytical apparatus.

In one aspect, the collection assembly may include a sampling member including a shaft extending from a connecting end to a sampling end. The connecting end of the sampling member may be coupled to the primary cap at the receiving portion.

In another aspect, the present disclosure provides device for collecting a biological sample. The device may include a tube defining a tube body and extending from an open end to a closed end. The tube body defines an exterior surface, an interior surface, and a tube volume bounded by the interior surface. The device may include a collection assembly at least partially engageable with the open end of the tube to define an engaged configuration. The collection assembly may include a primary cap defining a cap body and extending from a first end to a second end. The cap body defines an outer surface. The cap body may include a first portion including the first end and defining a receiving portion; a second portion including the second end, the second portion defining a cavity open to the second end; and an opening in the primary cap. The collection assembly may include a secondary cap having a sealing portion, the sealing portion sized to fit in the cavity of the primary cap. In the engaged configuration, the opening of the primary cap may be open to the tube volume. In one aspect, the present disclosure provides for a system including this device and an analytical apparatus. The connecting end of the sampling member may be coupled to the primary cap at the receiving portion.

In one aspect, the collection assembly may include a sampling member comprising a shaft extending from a connecting end to a sampling end.

In another aspect, the present disclosure provides a method of assaying a sample. The method may include steps including disengaging the collection assembly of the device of the present disclosure from the tube. The method may include collecting a sample from the patient with the sampling member. The method may include engaging the collection assembly with the tube to define a processing configuration so that the sample is sealed in the device. The method may include placing the device in the processing configuration in an analytical apparatus such that the closed end of the tube engages a portion of the analytical apparatus. The method may include removing the secondary cap of the device. The method may include analyzing the sample.

In one aspect, the present disclosure provides a method of assaying a sample. The method may include providing a tube defining a tube body and extending from an open end to a closed end. The tube body may define an exterior surface, an interior surface, and a tube volume bounded by the interior surface. Also provided may be a primary cap defining a removable cap body, the removable cap body being engageable with the open end of the tube. Further provided may be a sampling member connected to the cap body. The sampling member may include a hollow shaft extending from a connecting end to a sampling end, the connecting end being attached to the cap body. The sampling member may extend from the connecting end to the sampling end which is toward the closed end of the tube when the primary cap is engaged with the tube. Also provided may be a secondary cap which includes a sealing portion sized to fit in a cavity of the primary cap, such that when the secondary cap is removed from the primary cap, a continuous channel is defined through the hollow shaft of the sampling member from the primary cap to to the sampling end of the sampling member. In one step, the method may include removing the primary cap and sampling member to collect a sample with the sampling member, wherein the secondary cap remains sealed during sampling. The method may include replacing the primary cap and sampling member in the tube. The method may include removing the secondary cap and inserting liquid into the continuous channel of the hollow shaft, which liquid interacts with the sample on the sampling end. The method may include analyzing the sample.

### BRIEF DESCRIPTION OF THE DRAWINGS

Reference is made to the following drawings, wherein:

FIGS. 1A and 1B illustrate a collection device for biological samples in accordance with an aspect of the present disclosure in (A) perspective and (B) cross-sectional views;

FIGS. 2A-2E are views of a tube for a collection device constructed in accordance with an aspect of the present disclosure:

FIGS. 3A-3J are views of a primary cap for a collection device constructed in accordance with an aspect of the present disclosure:

FIGS. 4A-4F provide views of a secondary cap for a collection device constructed in accordance with an aspect of the present disclosure;

FIG. 5 illustrates steps from disengaging a collection assembly of the device from a tube as disclosed herein;

FIG. 6 illustrates steps from disengaging a secondary cap of the device from a primary cap:

FIG. 7 illustrates engaging a secondary cap of the device with the primary cap;

FIGS. 8A-8C are views of features of a tube, primary cap, and secondary cap constructed in accordance with the principles of the present disclosure;

FIG. 9 is a cross-sectional view of a cap structure of a collection device according to the principles of the present disclosure, engaged with a tube;

FIG. 10A is a cross-sectional view of another aspect of a device including a sampling member constructed in accordance with the principles of the present disclosure in an engaged configuration:

FIG. 10B is a cross-sectional view of the cap structure engaged with the tube of the device of FIG. 10A;

FIG. 10C is a view of another sampling member for a device constructed in accordance with the principles of the present disclosure:

FIGS. 11A-11C are views of features of a receiving portion of a collection assembly constructed in accordance with the principles of the present disclosure;

FIG. 12 flow chart of a sample collection and analysis workflow for a biological sample in accordance with a method of the present disclosure, relative to an existing workflow:

FIG. 13 is an example of an instruction sheet for a patient to follow when self-collecting a biological sample according to a method of the present disclosure:

FIG. 14 illustrates an array of devices of the present disclosure for testing in a multiplexed system:

FIG. 15 depicts an analysis assembly for processing samples in a workflow of the present disclosure;

FIG. 16 depicts further components usable in an analysis assembly for use with a method of the present disclosure;

FIG. 17 is a graph of sensitivity of a detection method in accordance with the present disclosure;

FIG. 18 is a graphical representation of raw test sample data collected according to methods disclosed herein; and

FIG. 19 is a flow chart of a sample collection and analysis workflow for a biological sample in accordance with the principles of the present disclosure.

### DETAILED DESCRIPTION

As used herein, the term “about,” means “approximately but not necessarily equal to,” and when used in the context of a numerical value or range set forth means a variation of 15%, or less, of the numerical value. For example, a value differing by ±15%, ±12%, ±10%, or ±5%, among others, would satisfy the definition of “about.”

As used herein, the term “analytical apparatus” refers to any piece of laboratory equipment involved in the processing of the sample contained in a device of the present disclosure. For example, an analytical apparatus may include a holder such as a tray or rack that supports any embodiment of the device, and may comprise features to secure projections on the base of the tube, as well as additional components such as a decapper, an agitator/shaker, a sonicator and/or a heating element.

As used herein, the term “sample” refers to biological material collected by using a device described herein, which can be from any source that can be engaged by a sampling member of such a device.

As used herein, a “patient” is an animal, particularly a mammal, and most particularly a human, from which a sample is or can be collected.

As used herein, the term “coupled” means attached to or fixed on or in, directly, or indirectly via intervening components.

Described herein are devices for sample collection, and methods for analysis of the same. These devices and methods allow for home collection by patients, provide a standardized collection swab and tube type, and can make use of PCR-ready, direct sample-lysing solutions to remove the need for traditional sample extraction.

The devices and methods provide a simplified and harmonized system of patient sample accessioning, one in which patient demographic information and sample tracking codes can be created, linked together, and securely uploaded into a digital warehouse, from which the testing laboratory can utilize an information management system and retrieve the information upon sample receipt. Sample accessioning may occur at the point of collection. The accessioning process may include the use of barcoding, such as using a two-dimensional barcoded sample collection vehicle as linkage of sample to patient health information (PHI). PHI may be input directly at the collection site and stored in a secure digital warehouse, enabling high-throughput electronic retrieval of PHI and accession by laboratory information management system (LIMS) at time of sample receipt.

The devices and methods provide rapid sample organization. Utilizing the rapid sample accessioning and LIMS processes, samples received can be racked rapidly for effectively immediate sample processing. The sample collection tubes can be scanned directly into the LIMS system, racked and subjected to automated cap removal or other method steps, followed by direct lysis of samples. This direct sample lysis may be from a dry-shipped swab, replacing traditional sample extraction. A variety of lysis buffers or solutions may be employed depending on their inhibitor tolerance, reproducibility, specificity and limit of detection (LOD).

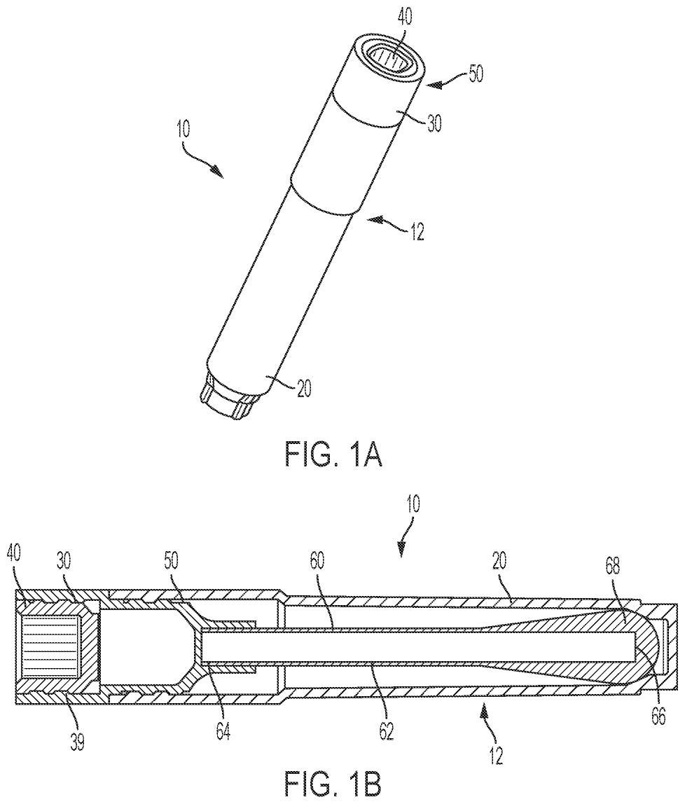

FIG. 1A illustrates a device 10 of the present disclosure. The device 10 includes a tube 20 and a collection assembly 50. The collection assembly 50, as can be seen in FIGS. 1A and 1B, includes a primary cap 30 and a secondary cap 40 which fits in primary cap 30. As defined herein, the collection assembly 50 is the primary cap 30, secondary cap 40, and any other component attached thereto, such as a sampling member 60, but not the tube 20. The collection assembly 50 is engageable with the open end 22 of the tube 20. Engaged configuration 12 is shown in FIG. 1A, wherein collection assembly 50 engages the tube 20. FIG. 1B provides a cross-sectional view showing the relationship of these components in the engaged configuration 12, including a sampling member 60, which includes shaft 62 and extends longitudinally away from the primary cap 30 from connected end 64 to sampling end 66, which may include an optional swab 68 or other sampling device.

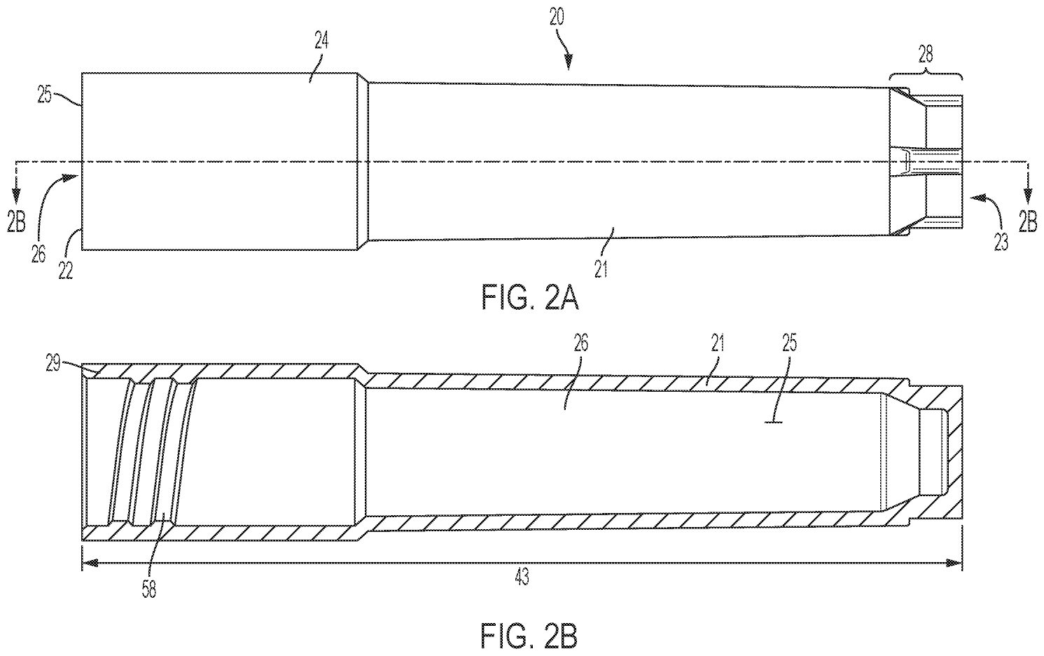

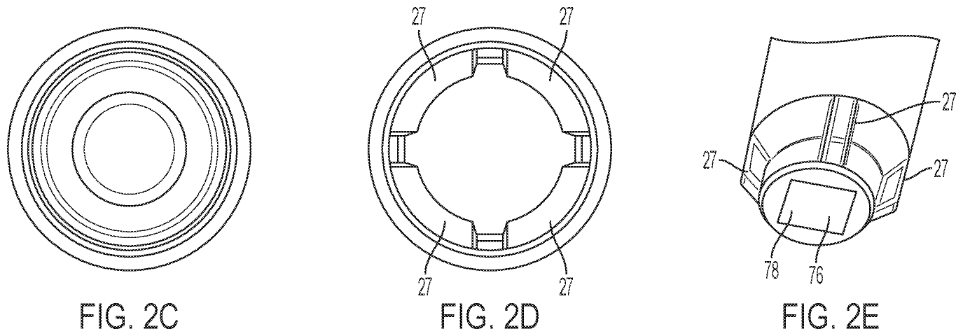

FIGS. 2A-2D provide views of the various features of the tube 20. The tube 20 defines tube body 21 and extends from open end 22 to closed end 23. Tube body 21 includes exterior surface 24 and interior surface 25 inside of the tube 20, which in turn defines a tube volume 26.

Closed end 23 of tube 20 may include a portion 28 defining at least one projection 27 thereupon. The projection 27 is capable of engagement with a component of an analytical apparatus. For example, the closed end 23 may include a plurality of projections 27, which may take the form of “fins.” The projections 27 may be spaced at substantially identical angles to one another on the exterior surface 24 of the tube 20, and may be constructed to have a mating fit with a tray or rack of the analytical apparatus in which the sample will be analyzed. In some embodiments, the projections or fins 27 do not extend beyond a diameter of about 3 mm to about 20 mm, or about 4 mm to about 15 mm, or about 5 mm to about 12 mm, or about 6 mm to about 10 mm, or about 9 mm of the tube 20 so that the exterior surface 24 of the tube 20 can contact the rack. On another portion of the tube 20, the tube may define a shoulder 29 which provides a point for contacting an analytical apparatus 80 that manipulates the device 10 and analyzes the sample contained therein.

In some aspects, the portion of the tray or rack into which the projections 27 of tube 20 fit may be constructed to allow for some initial rotational freedom of movement of the tube 20, such as up to about a one-quarter turn. When rotated beyond this, the tube 20 becomes “fixed” in position on the rack or tray. In some instances, the analytical apparatus may include an automatic decapping component, which may use a post structure to rotate the secondary cap 40 and uncap using rotational motion, that is, unscrew the secondary cap 40. When the decapping component exerts torque on the secondary cap 40, the remainder of device 10 will be held in place due to the fit of projections 27 in the tray or the rack, allowing the device 10 to be uncapped and the sample further processed. As will be described in more detail below, the interaction of the projections 27 with the analytical apparatus 80 may assist in keeping the primary cap 30 in position while the secondary cap 40 is removed.

FIG. 2E shows a closeup perspective view of the closed end 23 of tube 20. In this aspect, a central area 76 of the exterior surface 24 at the closed end 23 is flattened to accommodate the placement of a label 78. The central area 76 is more radially central than the projections 27 in this aspect. The label 78 may be a barcode. In the illustrated aspect, the printed area may be about 3 millimeters (mm) by 3 mm. Placement of the label 78 on the exterior surface 24 of the closed end 23 of tube 20 may allow for the device 10 to be read and identified by the analytical apparatus 80 even when decapped and in a rack.

The printing of barcodes and other labels 78 on the exterior surface 24 of the tube 20 is conducted with regard to manufacturing tolerances and in some aspects includes the provision of an area around the central area 76 to allow for consistent printing in the designated area. Printing may be achieved by applying an admixture to the surface at the desired position. For example, on a polypropylene tube, the admixture may include a colorant provided in a predetermined amount. In one aspect, the colorant may be for example Iriotec™, and it may be provided in a concentration of about 0.1-2.0%, or about 0.2-1.5%, or about 0.3-1.0%, or about 0.5-0.9%, or about 0.7% weight to weight (w/w). Other colorants known in the art may also be used. The finish of the central area 76, in the illustrated aspect, may also assist in rendering the surface printable, and in one aspect may have a roughness of satin finish in accordance with the ISO 213—N6 standard. It will be appreciated that the area for applying a label may be anywhere on the tube 20 or the collection assembly 50, or both, and need not be restricted to a closed end of the tube 20.

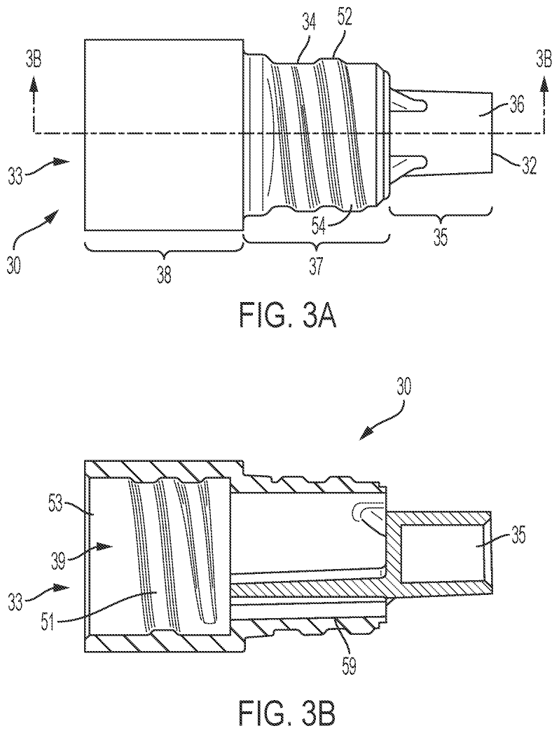

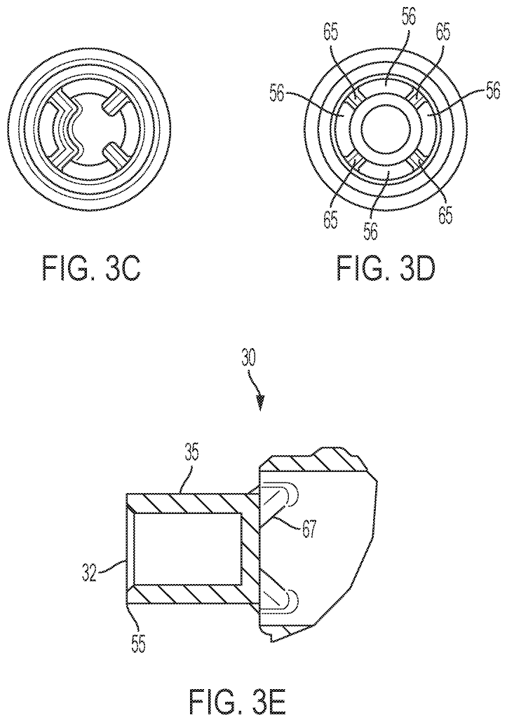

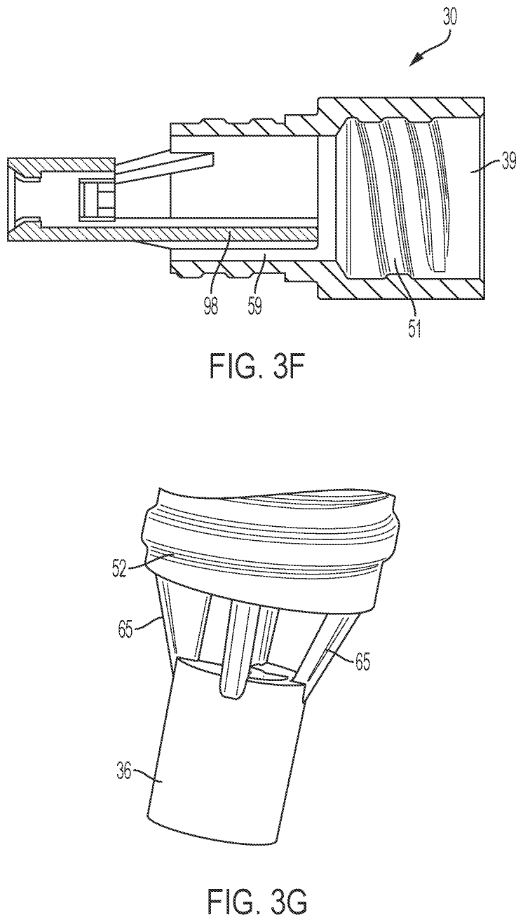

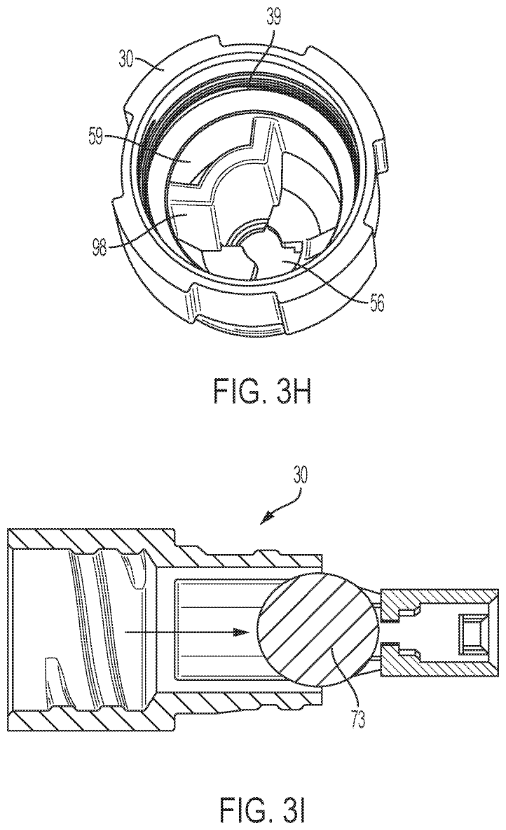

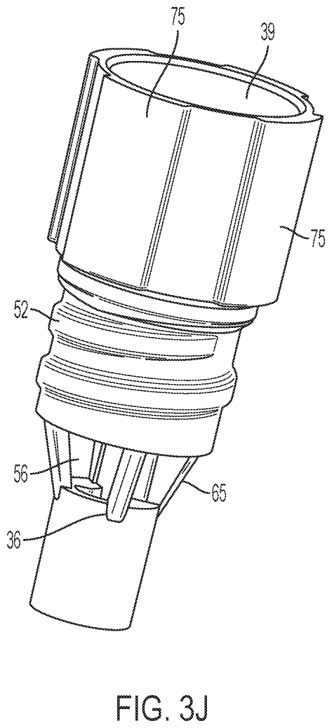

FIG. 3A-3E depict the primary cap 30 of device 10. The primary cap 30 defines cap body 31, and extends from first end 32 to second end 33. The cap body 31 defines an outer surface 34. The cap body 31 includes a first portion 35 which includes the first end 32. The first portion 35 includes a receiving portion 36 which can receive, for example, a sampling member 60. In some aspects, the receiving portion 36 may include a chamfer 55 to better accommodate the sampling member 60.

The cap body 31 also defines a second portion 38 which includes a cavity 39 for the secondary cap 40. The cap body 31 may have a middle portion 37 located between the first portion 35 and the second portion 38, such as between them in a longitudinal dimension. A thread 54 may be formed on an outer surface 34 of the primary cap 30, such as on the middle portion 37.

To maintain sample integrity, the primary cap 30 may form an airtight seal with the open end 22 of the tube 20, and the secondary cap 40 may form an airtight seal with the primary cap 30. In some aspects, the seal may be a sterile seal. Fluid flow through the primary cap 30 and into the tube volume 26 during sample processing may prove difficult. For this reason, the primary cap 30 may be constructed with more than one opening. In the illustrated aspect of FIGS. 3A-3E, a first opening 56 may be formed through the primary cap 30 in order to allow liquid deposited inside of the primary cap 30 to make its way into tube 20, where it may interact with the collected sample.

The opening 56 may be located at a substantially central portion 57 of the primary cap 30, relative to the longitudinal axis. As seen in FIG. 3E, the portion of the primary cap 30 at which the first opening 56 is defined may have a shape 67 similar to a funnel (see, for example, FIG. 3G), such that liquid is made to flow by gravity through the primary cap 30 and into the tube 20 when the device 10 is placed in the analytical apparatus 80, in which the device 10 is held upright. The opening 56 may be defined by one or more spaces between supports 65 which secure the receiving portion 35 of primary cap 30 in a substantially spoke-and-axle type of configuration, as seen in the end view of primary cap 30 shown in FIG. 3D. FIG. 31 illustrates the relative size of a drop of liquid 73 that can pass onto the sample from primary cap 30 due to this funnel-shaped construction.

A second opening may be defined through the primary cap 30 in the form of channel 59. Channel 59 may function as a gas or air vent. Details of one such channel 59 can be seen in FIGS. 3B, 3F, 3G, and 3H. In the illustrated aspect, the channel 59 allows evacuation of air out of tube 20 through primary cap 30, when liquid is added through the opening 56 in the primary cap 30 and air from within the tube 20 is displaced. The primary cap 30 may include one or more openings 56 through which fluid may flow. The openings 56 may be used to direct the flow of liquid, such as to the interior perimeter of the tube, the exterior perimeter of the shaft 62, or the interior perimeter of the shaft 62 in embodiments where shaft 62 has a hollow center that extends to the sampling end 66. In such cases liquid may descend to contact the swab 68 or other sampling component (such as a punch, needle, microneedle, scalpel, curved scalpel, scoop, wire loop, blade loop or tweezers) through the center of the hollow shaft 62. In some embodiments pressurized gas (optionally containing aerosolized reagents) may be used in addition to or instead of liquid reagents.

As can be seen in in FIG. 3F, the height of the liquid opening 56 is lower than the channel opening 59, so that when liquid is introduced, the liquid flows through the opening 56 into tube 20, but not through channel 59. That is, the channel wall 98 surrounding and defining channel 59 extends longitudinally into the interior of the primary cap 30, causing the channel 59 to terminate closer to the second end 33 than does opening 56. In some embodiments, the channel 59 is located in a peripheral portion 63 of the primary cap 30, rather than in a central portion 57, where the first opening 56 may be located.

This construction generalizes liquid handling. The tips of the liquid handler may be positioned over the center portion 57, of the primary cap 30, not offset, which accommodates non-contact liquid addition using any liquid handling system. The height of the channel 59 relative to the first opening 56 allows for air to escape from the tube 20 without introduction of liquid from the liquid handler. In some embodiments, the opening 56 may be angled to overcome the surface tension of added liquid (see FIGS. 3F and 3G). In one aspect, the angle of supports 65 defining a portion of openings 56 may be about 0 to about 20 degrees, or about 2.5 degrees to about 17.5 degrees, or about 5 degrees to about 15 degrees, or about 7.5 degrees to about 12.5 degrees, or about 10 degrees, or about 15 degrees, or about 20 degrees, relative to a longitudinal axis of the primary cap 30. If desired, the temperature of the device 10 can be raised to assist with liquid movement. In some embodiments, liquid, such as a lysis buffer, RT-PCR master mix, or a sample diluent, can flow down an exterior of the shaft 62 of sampling member 60 if the shaft 62 is solid, and through the shaft 62 if the shaft is hollow, onto swab 68 if included, facilitating mixture of liquid and sample, thereby advancing the analytical workflow.

In another aspect, the primary cap 30 may include different relative placements of the liquid opening 56 and the air channel 59. For instance, the channel 59 may be formed in a center portion 57 of the primary cap 30, and the opening 56 formed in an offset or peripheral portion 63.

In one example, an array of an analytical apparatus 80 for use with the device 10 of the present disclosure may have a small amount of space between devices. In one example, the tubes may be spaced 9 mm center-to-center. In order to keep the space within the primary cap 30 large enough to make fluid flow efficient and thorough, the primary cap 30 may be manufactured with a relatively thin wall. As seen in FIG. 3J, features such as supports 75 can be added to extend the outer surface 34 of the primary cap 30 in certain positions, allowing it to have a thin profile overall but still withstand squeezing or twisting by the patient or by components of the analytical apparatus 80. In some aspects, the wall of the primary cap may be less than 0.5 mm thick, or about 0.4 mm thick, in the portions lacking supports 75. The addition of supports 75 may allow for the torque for inserting secondary cap 40 to be consistent during manufacture and during laboratory handling in the analytical step.

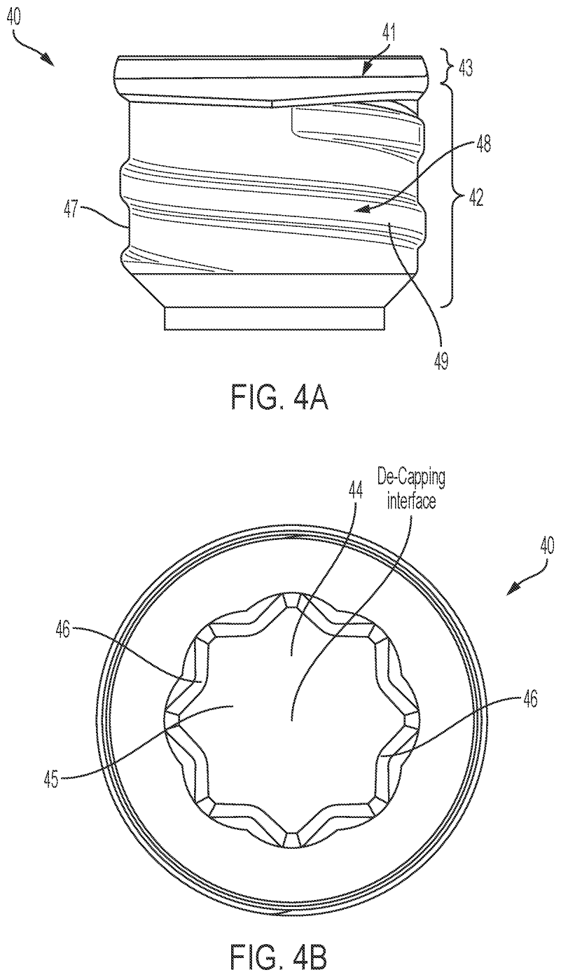

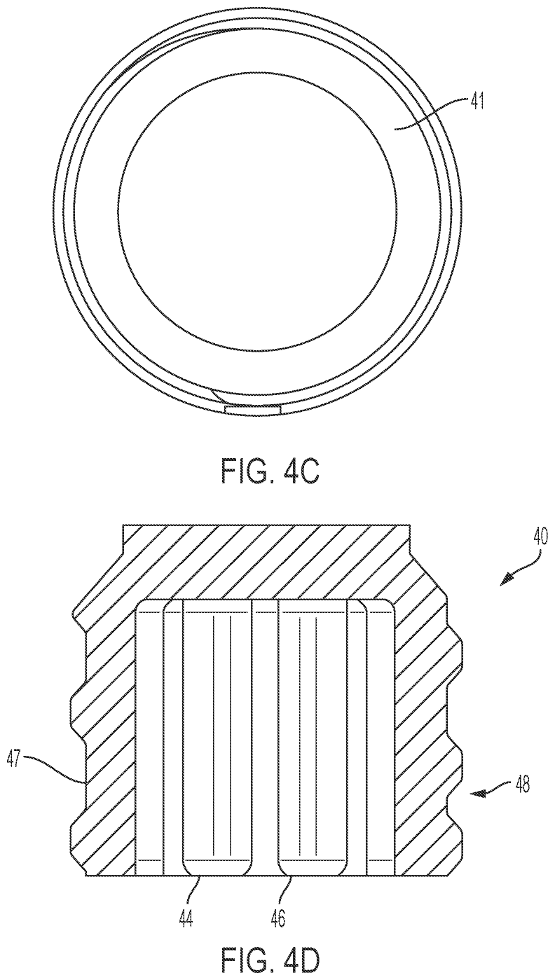

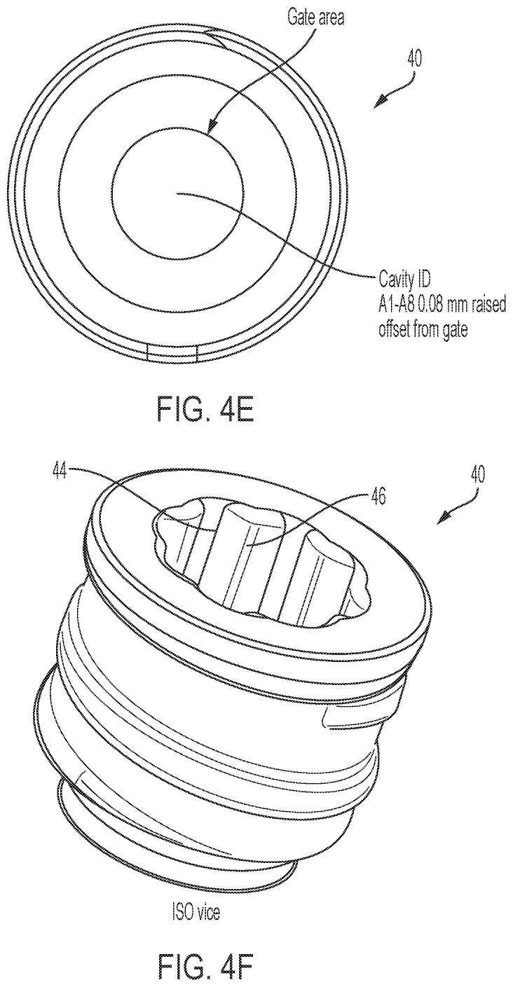

FIGS. 4A-4F illustrate the secondary cap 40. The secondary cap 40 includes a sealing portion 41 providing a seal closing off the primary cap 30. As discussed above, the seal may be an airtight seal, and in some embodiments, a sterile seal. The sealing portion 41 is sized to fit in the cavity 39 of primary cap 31. The secondary cap 40 includes a proximal portion 42, which includes the sealing portion 41, and a distal portion 43. The distal portion 43 may include an interface 44 for engaging a portion of an analytical apparatus or other processing machinery. For example, interface 44 may provide the capability for engaging a decapping assembly, which may remove the secondary cap 40 from the remainder of device 10 automatically, and in some aspects may also replace the secondary cap 40 after one or more reagents are added to the device 10. In one aspect, the interface 44 may be formed as a depression 45 where a protrusion of the decapping assembly can insert and provide a connection that enables decapping. As seen in FIGS. 4B and 4F, the interface 44 may include projections 46 extending into depression 45 to form a mating fit with the decapping assembly.

As seen in FIGS. 4A and 4F, an external surface 47 of the secondary cap 40 may include a thread 48. The thread 48 provides for sealing engagement with a corresponding thread 51 formed on a surface of primary cap 30 that surrounds at least a portion of cavity 39. The direction of the thread 48 on the secondary cap 40 matches the direction of a thread 51 formed in cavity 39 of the primary cap 30, which receives the secondary cap 40. The threads 48 and 51 can both be clockwise or counterclockwise threads. The threads 48 and 51 are formed in a direction opposite that of thread 52 formed on the outer surface 34 of primary cap 30, and that of thread 58 formed on the interior surface 25 of tube 20, which receives the thread 52 of primary cap 30. For instance, when threads 48 and 51 are formed to engage when rotated counterclockwise, then threads 58 and 52 are formed to engage when rotated clockwise. When threads 48 and 51 are formed to engage when rotated clockwise, threads 58 and 52 are likewised formed to engage when rotated counterclockwise.

Referring now to FIG. 1B, the collection assembly 50 of device 10 may include a sampling member 60. The sampling member may include an elongate member such as a shaft 62, which extends from a connecting end 64 to a sampling end 66. The connecting end 64 may be a fixed end, attached to the primary cap 30. The sampling end 66 may be a free end, which, when the collection assembly 50 is engaged with the tube 20 in the engaged configuration 12, The sampling end 66 of the sampling member 60 is provided according to the type of sample to be obtained. For example, in a device 10 intended for collection of a nasal sample, a swab 68 may be disposed at the sampling end 66.

The swab 68 may, in one aspect, be made of a polymer, and in another aspect may be made of a natural material such as cotton. In one aspect, the swab 68 may be made of polyester. In one aspect, a polymer swab 68 may be made with a flocked material. In another aspect, the swab 68 may be made of a spun polymer.

The shaft 62 of sampling member is provided in accordance with the type of sample to be obtained. For instance, in a COVID-19 test as taught herein, a shaft 62 is provided having a length for obtaining a nasal sample. In some embodiments, the length for a sampling member 60 within the tube volume 26 from the end of the receiving portion 36 of the primary cap 40 to the end of the interior surface 25 of the tube 20 may be about 20-50 millimeters, and in some embodiments, about 20-40 millimeters, and in one embodiment about 36 millimeters (mm). In other embodiments, the shaft 62 may be any length depending on the sample being collected. The shaft 62 may be of a hollow construction in one aspect. In another aspect, the shaft 62 of the sampling member 60 is solid.

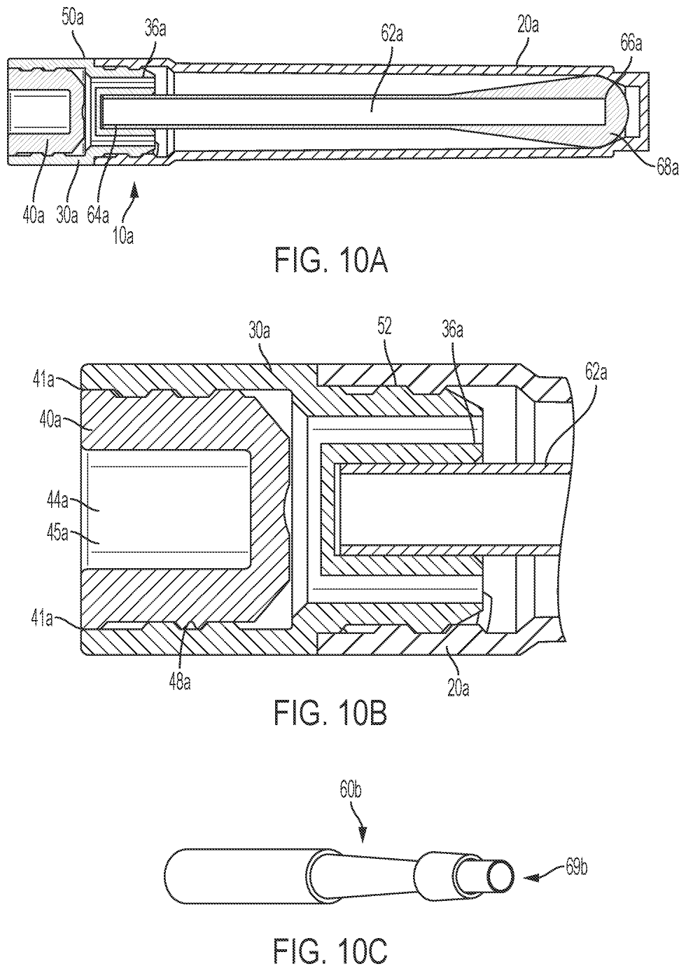

It will be appreciated that a sampling member 60 will be provided with a shaft 62 and a sampling end 66 in accordance with the intended sample type for the device 10. For instance, when the device 10 is a coronavirus testing device for a shallow nasal collection, the shaft 62 may be about 36 mm long, and the sampling end 66 may include a swab 68 made of a polymer. When device 10 is intended for a nasopharyngeal COVID-19 test, the shaft 62 may be longer (such as up to 170 mm), and provided with a corresponding longer tube 20. In other aspects, the sampling end 66, or the full sampling member 60, may be other structures and features. For instance, when the sample is a portion of a eukaryotic organism (a tissue, an organ, a leaf, a stem, and so forth), the sampling member 60b may be a punch 69b, as seen in FIG. 10C. In this aspect, the sample (such as a plant part) would be punched, and the obtained sample would remain in the tube of the device. In other aspects, the sampling end or sampling member may include needles, microneedles, scalpels, curved scalpels, scoops, wire loops, blade loops, tweezers and other components.

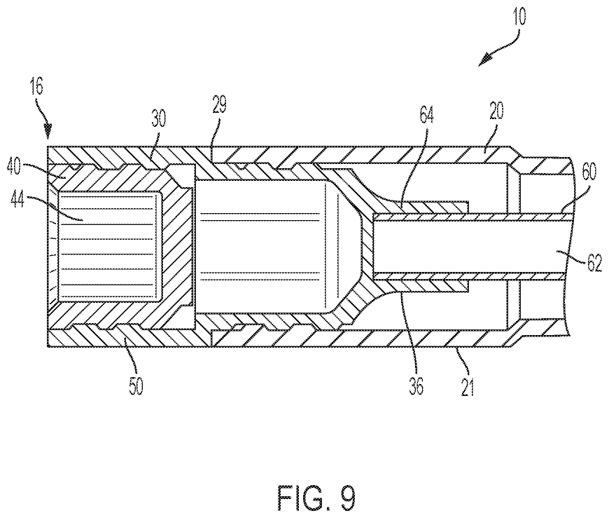

The connecting end 64 of sampling member 60 may be coupled to the primary cap 30. In some embodiments, the primary cap 30 may include a receiving portion 36. As shown in FIGS. 1B and 1n FIG. 9B, the receiving portion 36 may at least partially or completely surround the shaft 62 for a portion of its length, in some aspects including the connecting end 64.

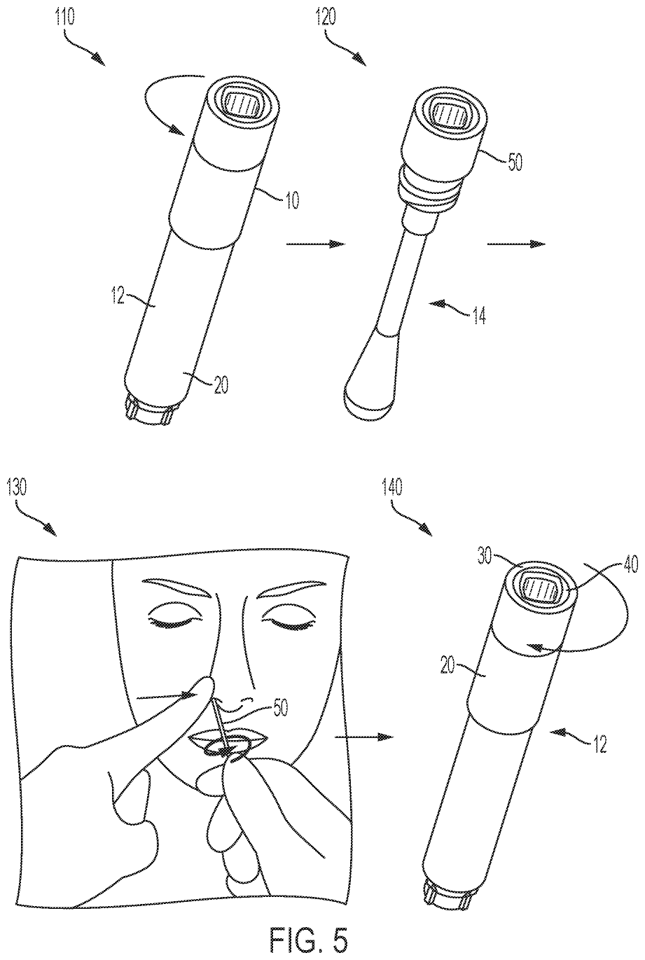

FIG. 5 illustrates an example of steps in a patient's sample collection workflow. Step 110 shows a device 10 wherein the collection assembly 50 is engaged with the tube 20. The device in this illustration is in the engaged configuration 12, and to collect a sample, the collection assembly 50 is disengaged from the tube 20, to attain disengaged configuration 16 in step 120. In the illustrated device 10, a user (such as a patient self-administering a collection) can unscrew the primary cap 30 from the tube 20, as illustrated in the counterclockwise direction. The primary cap 30 and the secondary cap 40 remain engaged when the collection assembly 50 is disengaged from the tube 20. Now, a user can collect a sample, such as a nasal swab as shown in step 130. When collection is complete, in step 140, the collection assembly 50 is then screwed back onto the tube 20, returning the device 10 to its engaged configuration 12, and sealing the sample away from the environment. The sample, within device 10, can now be shipped off and subjected to analysis. Additional types of samples may be collected using a similar work flow that includes disengaging the collection assembly 50 from the tube 20 with the primary cap 30 and second cap 40 remaining engaged. A sample of any type may be collected and the collection assembly 50 returned to the engaged configuration 12.

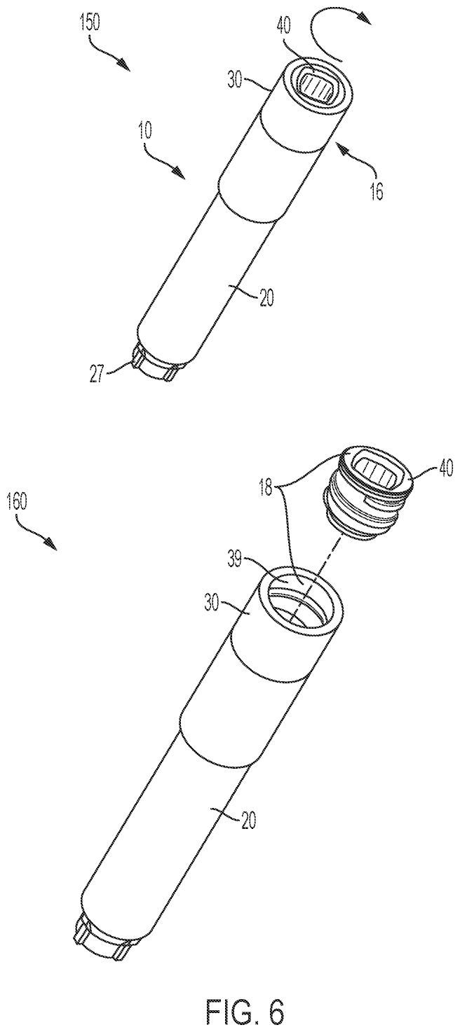

FIG. 6 illustrates an example of an initial step in an analytics workflow. In step 150, the device 10, which contains a collected sample, has primary cap 30 in an engaged configuration 16 with the secondary cap 40. To disengage, the secondary cap 40 is rotated, in the illustrated aspect clockwise, to separate the secondary cap 40 from the primary cap 30. This is an opposite rotational direction from the direction in which the primary cap 30 is rotated when it is detached from the tube 20, as the thread 52 formed on the outer surface 34 of primary cap 30 is formed with a first handedness (that is, direction in which the thread rotates about the longitudinal axis around which is formed) and the thread 48 formed on the secondary cap 40 is formed with a second handedness opposite the first handedness. Thus, clockwise rotation of the collection assembly 50 relative to the tube 20, when the tube 20 is held in a fixed position, will only disengage the secondary cap 40 from the primary cap 30, and in the counterclockwise direction, the collection assembly 50 as a whole will be removed from the tube 20.

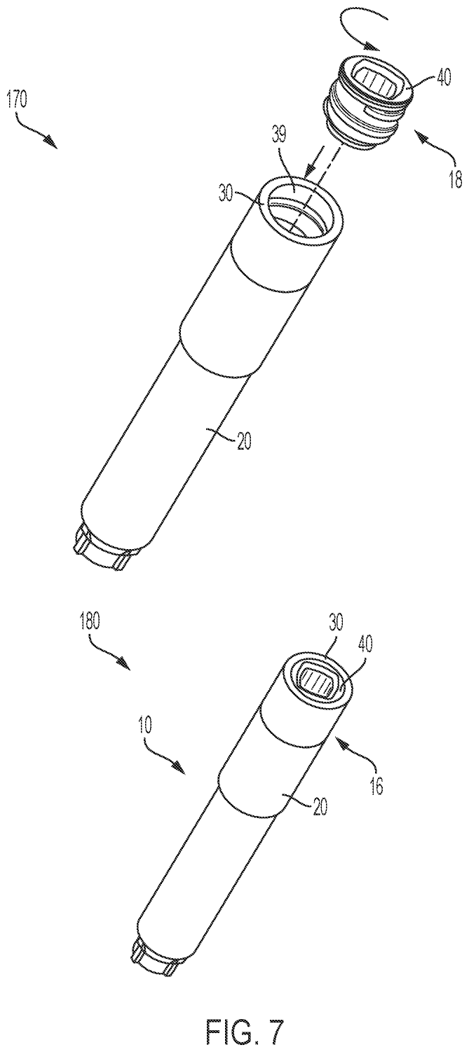

FIG. 7 illustrates steps in reversing the process of FIG. 6 within the analytics workflow. In step 170, in one aspect after a liquid for processing has been added to the device 10 via the primary cap 30, the secondary cap 40 is placed into the cavity 39 of primary cap 30 and rotated in the opposite direction (in the illustrated device, counterclockwise) such that the secondary cap 40 is flush with the top of primary cap 30.

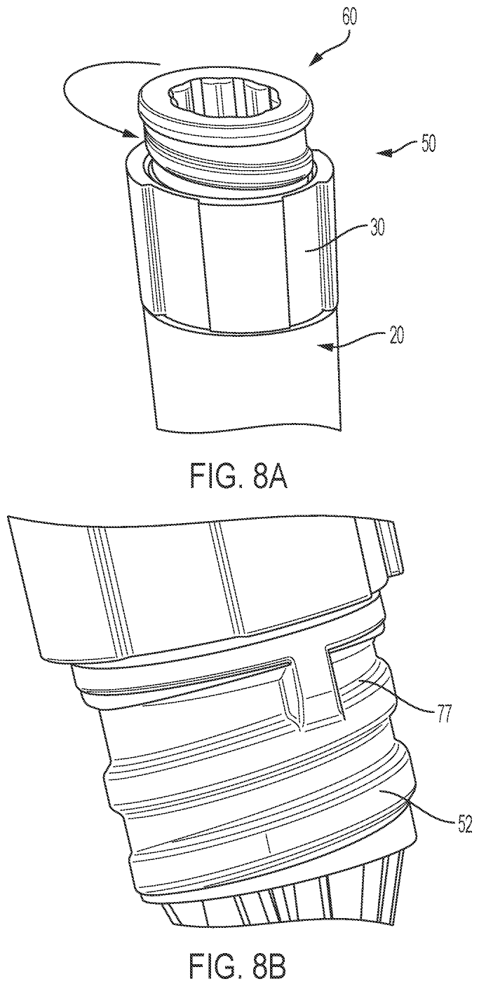

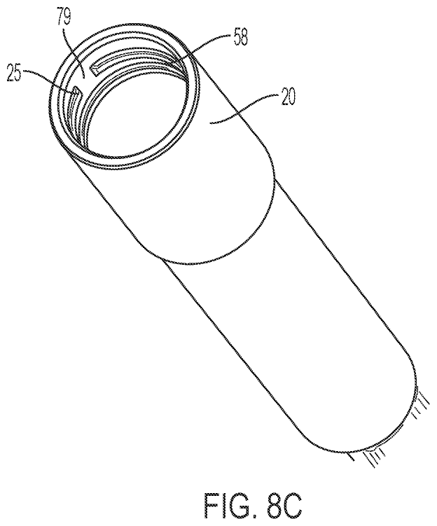

FIGS. 8A-8C show one option for the interaction between the collection assembly 50 and the tube 20 of device 10. In the example illustrated in FIG. 8A, the secondary cap 40 has a counterclockwise thread 48 formed on its external surface 47, and rotation will cause the collection assembly 50 to detach from the tube 20 when in the analytical apparatus 80 if these components are not secured.

FIG. 8B shows a protrusion 77 formed on primary cap 30 on a portion of thread 52 close to the second end 33, which fits with a slot 79 on In another aspect, the tube 20 may include a protrusion, and the primary cap 30 include a slot for fitting the protrusion. When the primary cap 30 is rotated into the tube 20 such that the device 10 adopts its engaged configuration 12, the protrusion 77 fits into slot 79, and the two components stop rotating relative to one another until intentionally displaced.

FIG. 9 illustrates interaction surfaces between the primary cap 30 and the second cap 40, and the primary cap 30 and the tube 20. The interface 44 on the secondary cap 40 may provide an interaction surface with decapping equipment, and in the illustrated aspect does so with a nonstandard connection measuring about 3.5 mm by 3.5 mm. In another aspect, when the primary cap 30 is engaged with the secondary cap 40 in an engaged configuration 16, the design of the sealing portion 41 of the is such that the seal functions as a plug seal. The overlap at this sealing portion 41 may be nominal, such as less than about 0.1 mm, or about 0.08 mm. At the same time, the thread 48 of the secondary cap 40 may be formed with a 2 mm pitch and with 0.2 mm overlap with the corresponding thread 51 formed surrounding the cavity 39 of primary cap 30.

FIGS. 10A and 10B illustrate a different construction of the caps 30a and 40a of a device 10a. The general construction of device 10a is fundamentally the same as that of device 10 of FIGS. 1-8, but in this case, the receiving portion 36a for the shaft 62a of sampling member 60a is formed facing into the cap body 31 of primary cap 30, rather than extending outwardly therefrom (see projections 65 supporting receiving portion 36 in FIG. 3A, compared for FIGS. 10A and 10B.)

The sampling member 60 may be fit into the primary cap 30 in a number of ways. In one aspect, the shaft 62 is fit into the receiving portion 36 by a push fit or a friction fit, wherein the size an aperture or cavity of the receiving portion 36 is sized such that it locks the shaft 62 into place. Optionally, the shaft 62 can be secured in the receiving portion 36 by a glue or an adhesive, or in another conventional way to join two separately-formed pieces.

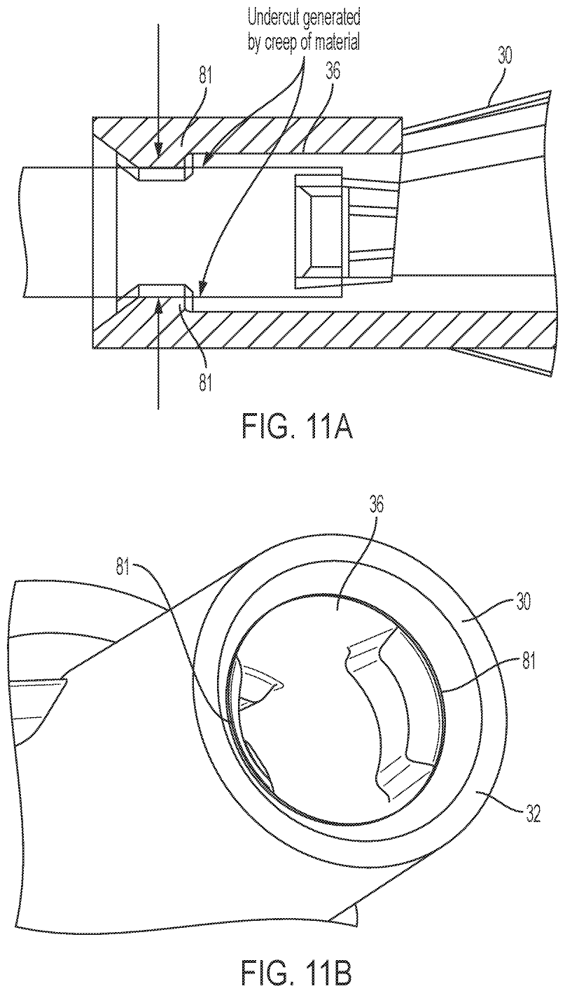

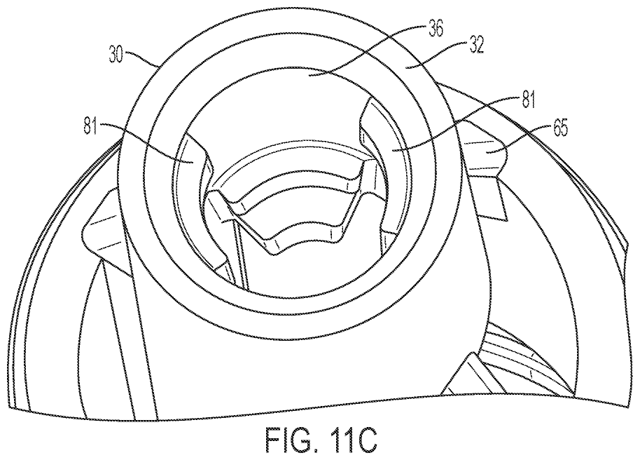

In another aspect, and as particularly illustrated in FIGS. 11A-11C, the fit of the shaft 62 into the receiving portion 36 involves utilizing the creep properties of the materials, such as plastics, to secure the sampling member 60. Such materials may include polypropylenes (PP), polyethylenes (PE), and other polymer materials with similar properties. For instance, the receiving portion 36 may include one or more tabs or grips 81 which contact the shaft 62 of the sampling member 60 at or near the connecting end 64. In one example, particularly illustrated in FIG. 11B and FIG. 1 IC, the primary cap 30 may include two grips 81, which have an arc shape and which are firmed directly across from one another on an interior surface of the receiving portion 36. Other numbers (3, 4, or more) and shapes of grips are possible.

When the shaft 62 is first inserted into the receiving portion 36, the geometry of the grips 81 allows for a press fit connection between the components of the collection assembly 50. As illustrated in FIG. 11A, the grips 81 are slightly undercut to account for material creep. Over time, the plastic may relax and creep in under the press fit geometry and will generate resistance of extraction of the sampling member 60 from the primary cap 30.

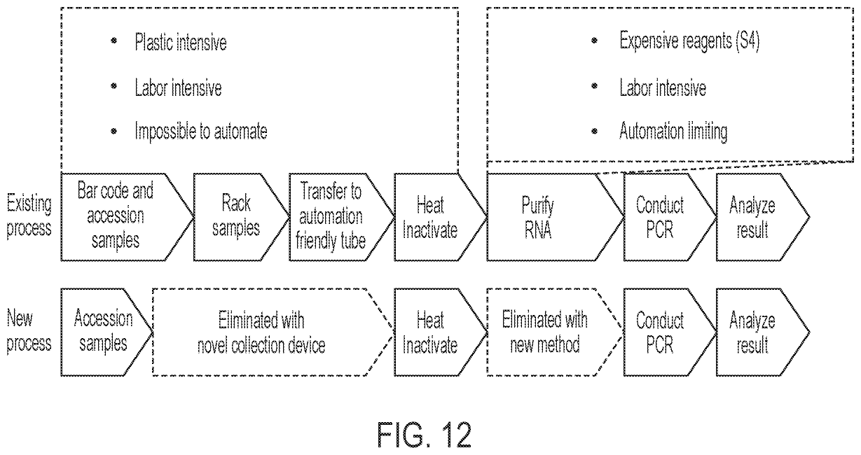

FIG. 12 compares a previous workflow alongside the method disclosed herein using a PCR assay as an example. Similar advantages may be obtained using different assays, automated and manual processes. In an embodiment of the analysis workflow as presently practiced, a sampling device is bar coded, and a sample acquired. The samples are then racked, transferred to an automation-compatible tube, and heat inactivated. The RNA is then purified, PCR conducted, and the results analyzed. The present process is labor intensive, generates a large amount of plastic waste, and is difficult or impossible to automate. The added steps of purifying RNA adds a labor intensive step using expensive reagents, and is a limiting step where automation is concerned.

In contrast, as shown in the lower panel of FIG. 12, the process begins with sample accession, as the devices 10 of the present disclosure are provided to the customer, laboratory, point of sampling, or patient, in pre-barcoded form. The use of device 10 eliminates the racking and transfer steps, and when loaded into an analytical apparatus 80, the samples can proceed to heat inactivation, saving time and labor. Further, RNA extraction is not necessary; the PCR workflow (optionally including a reverse transcription step) can begin immediately, after which analysis of the sample occurs.

Although in some aspects, an RNA extraction-free (that is, direct to PCR) workflow may be desired, particularly in the context of a test for COVID-19, when the device 10 is used in conjunction with the analysis of another type of sample, the automation conditions may differ. For example, in a sample type for which the presence of RNA decreases accuracy or sensitivity, the liquid handler which adds an assay buffer may introduce an RNase to each device 10. The assay conditions, including the components and amounts of buffer, can be adjusted accordingly by the person of skill in the art.

In one aspect, the present disclosure provides a device 10 which can be used to perform a method of assaying a sample. The method may include disengaging a collection assembly 50 of a device 10 as described throughout this disclosure, in some aspects by providing a torque to the primary cap 30 of the collection assembly 50, sufficient to disengage protrusion 77 of the primary cap 30 from the slot 79 of tube 20, if present, thereby unscrewing the collection assembly 50 from the open end 22 of tube 20. The primary cap 30 and the secondary cap 40 remain engaged when the collection assembly 50 is removed from the tube 20. The sample is then collected from the patient or biological test subject by use of the sampling member 60. The device 10 is returned to the engaged configuration 12 by engaging the collection member 50, now harboring a sample, with the open end 22 of tube 20. The sample then remains sealed within the tube volume 26 of tube 20. This defines a processing configuration of the device 10, as the next instance in when any of the primary cap 30, secondary cap 40, and tube 20 will be disengaged from one another will be during sample analysis.

The device 10 is then conveyed from the patient or biological test subject to the testing facility, whether by shipping, or hand delivery at a medical facility, or the like. In some aspects, the device 10 may be conveyed to the testing facility at room temperature. The device 10 is placed into an analytical apparatus 80 such that the closed end 23 of the tube 20 engages a portion of the analytical apparatus (such as a tray or a rack). If the tube 20 is formed with projections 27, these are suitable portions for contacting the analytical apparatus 80.

The secondary cap 40 is then removed, such as by unscrewing it from the primary cap 30 with an opposite rotational direction from the unscrewing action of the collection assembly 50 from the tube 20 mentioned in a previous step. This unscrewing step may be an automated step, using standard or bespoke laboratory equipment to engage the secondary cap 40, and providing rotational motion. This converts the device to its disengaged configuration 18. Finally, the sample is analyzed. This may be done by adding liquid to the tube 20 through the primary cap 30, now that the secondary cap 40 has been removed. The method may include resealing the secondary cap onto the primary cap 30, and eluting the sample from the collection assembly 50 using the liquid. The analysis may include heating the sample, and conducting a polymerase chain reaction (PCR). The analysis can be conducted in the context of a manual analysis, a low-throughput workflow, or a high-throughput workflow.

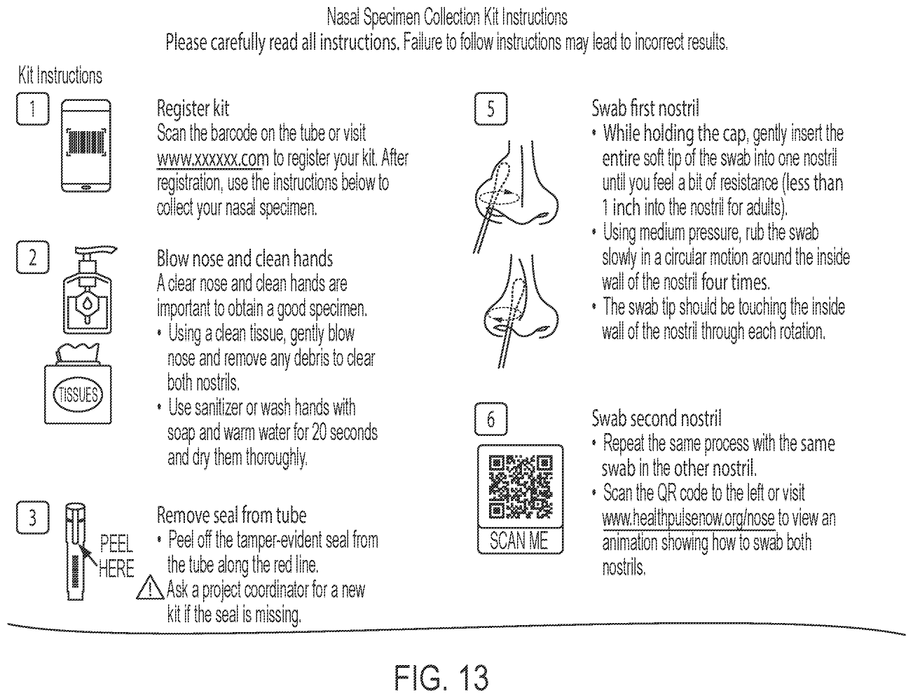

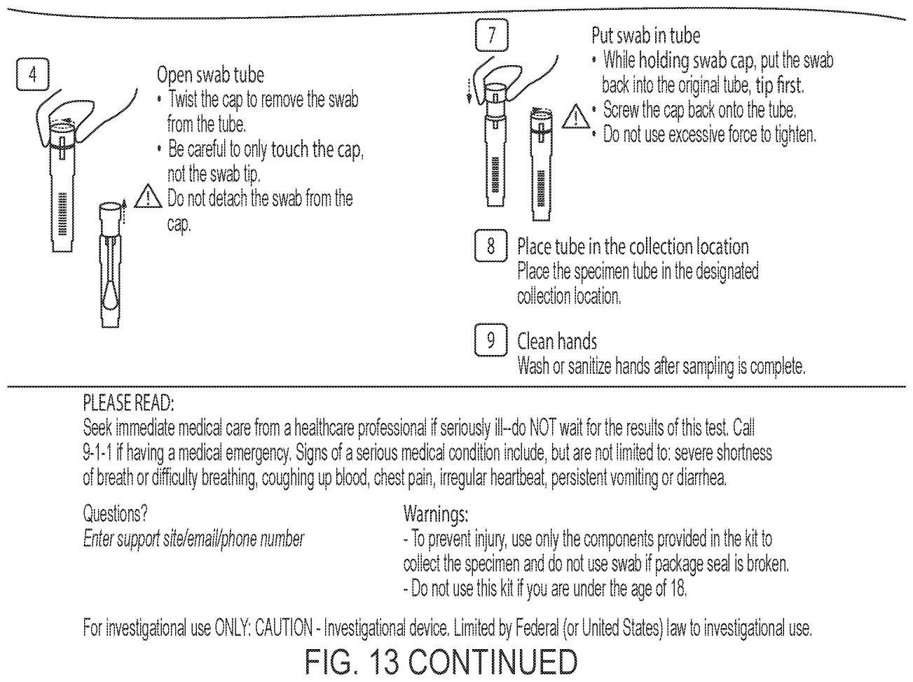

FIG. 13 depicts an example of instructions that can be provided to a patient for self-collection. The instructions will vary depending on the type of sample to be collected. The instructions may include descriptions of how to associate the identity of the patient with the sample by using a phone app to scan the barcode; how to sanitize the collection site, such as the nares region for administration of the test and collection of the sample; how to handle the various components of the device 10 in order to reliably collect a sample in such a way as to achieve the most reliable test result, and how to collect the sample and ship it to the testing facility. The instructions may be packaged with the device 10, or may be provided online, such as for access by a mobile device. The kit provided may include a sanitizing solution to standardize the sanitization step.

Self-collection by the patient saves on time and on costs. Rather than take the time of a skilled practitioner at a medical facility, the patient quickly and inexpensively administers the test and collects the sample at home. The collection assembly 50 and tube 20 structure of the device 10 as disclosed herein allows for a single exposure event of the sampling member 60 to the environment from the time the device 10 is manufactured to the time the sample is analyzed, while allowing for no exposure to the portion where the assay liquid is introduced (that is, the cavity 39 of primary cap 30, which is sealed from the environment by secondary cap 40). Self-collection may be facilitated because the device 10 is shipped to, and shipped back by, the patient with no liquid present in the device 10.

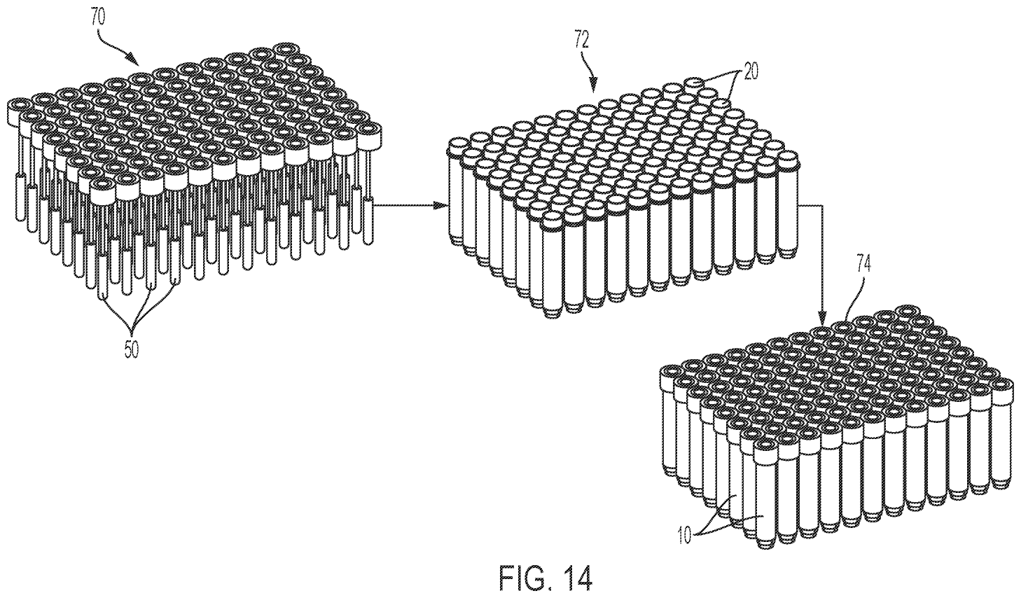

In one aspect, the present disclosure provides a method featuring scalable multiplexing of sample analysis. FIG. 14 shows an array 70 of collection assemblies 50 which have been decapped, automatically, from their associated tubes 20. Many multiples of devices 10 can be arranged in an array 72 for a multiplex assay. The tube 20 of the device 10 can be designed to interface with existing equipment or with a bespoke tube rack, allowing for simultaneous processing of numerous samples under identical conditions.

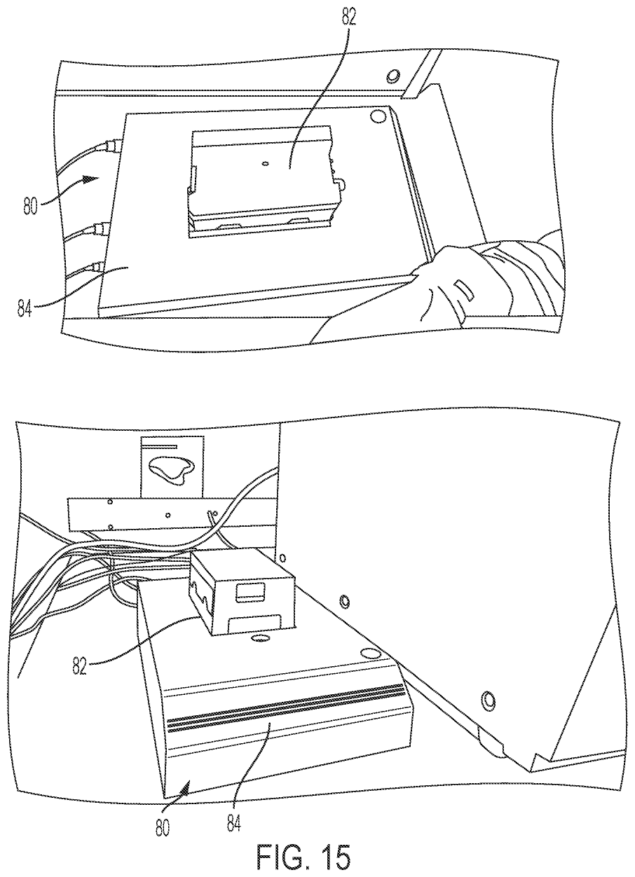

An exemplary analytical apparatus 80 for analyzing samples collected by device 10 can be seen in FIG. 15. The analytical apparatus 80 may include (that is, comprise or consist of) a specimen rack 82, which may be assembled onto flatbed scanner 84. The specimen rack 82 may be sized to fit a 96-well plate, or a 384-well plate, or other applicable size. The scanner 84 may be capable of reading visible light, infrared, and/or ultraviolet, and may have luminescence and fluorescence modes. The analytical apparatus 80 may also include other equipment, such as a decapper, an agitator/shaker, a sonicator, and a heating element, among others.

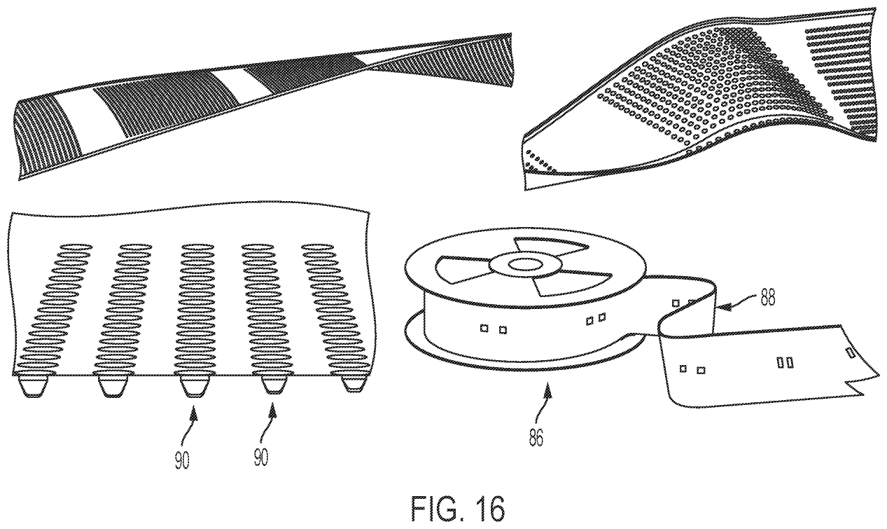

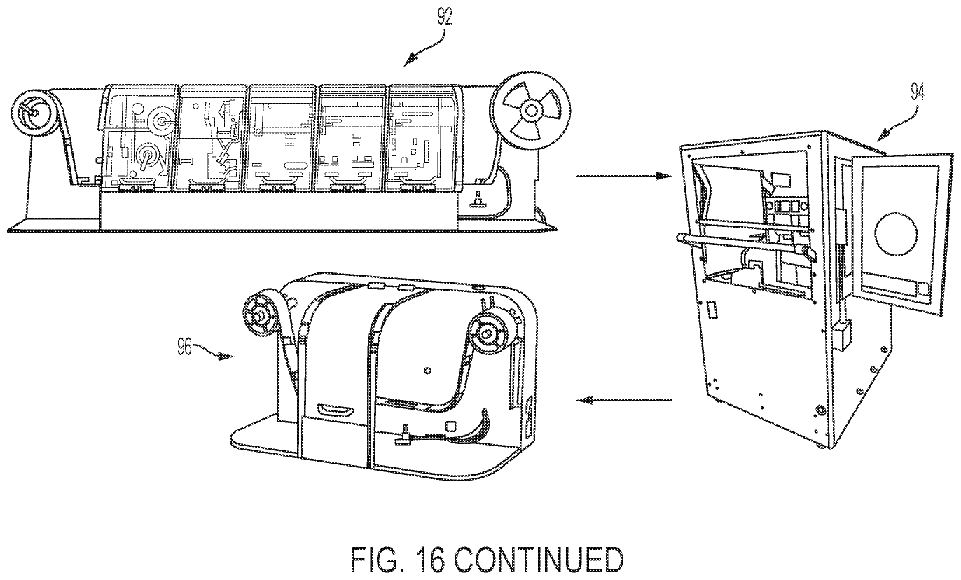

In one aspect, the devices 10 described herein, and their attendant methods, can be employed on an analytical apparatus which includes devices with a very high throughput. FIG. 16 illustrates components of an analytical apparatus including such devices. One such approach may include using tape-based array workflows that can process tens of thousands of samples per day per instrument. See, for instance, tape 88 held on reel 86 in FIG. 16. A closer view of tape 88 illustrates numerous wells 90 having a small capacity (about 0.1 to about 10 microliters, or about 1 to about 5 microliters) but which nonetheless can hold sufficient sample to be analyzed by the downstream amplification assay. A portion of the liquid derived from the samples collected by devices 10 may be transferred to the wells 90 of tape 88.

Further components of the high throughput testing system and method may include reverse transcription-polymerase chain reaction (RT-PCR) for nucleic acid detection and amplification, and data analysis. Both real-time PCR and end-point analysis can be used to return reliable results for a large volume of samples in a small amount of time. The tape 88 can be submitted to conditions suited to either PCR workflow.

Analytical assemblies including 96-well, 384-well, and 1536-well PCR systems, as well as ultra high throughput systems such as the Douglas Nexar tape array platform 92, can be used in the methods described herein. The tape 88 can be transferred to a high-capacity cycler 94, such as a Hydrocycler, for endpoint PCR. The present devices enable methods that allow for testing of 75,000 samples per day, or greater than 300.000 samples per day, at a laboratory cost of under five dollars per test, with the sensitivity and selectivity of a RT-PCR assay (limit of detection of under 4000 pathogen copies per sample, in the case of a viral pathogen). The results may then be read by reader %, which in one example measures fluorescence.

Depending on the sample type, other equipment, solutions, and so forth may make up part of the analytical apparatus 80. For instance, if cell walls need to be disrupted prior to sample analysis but after collection, a sonicator may be employed. Various enzymes or solutions that eliminate or reduce signal-impeding molecules, including but not limited to oils, sodium hydroxide lysis solutions, hydrogels, and detergents, may be introduced in an additional step via the liquid handler.

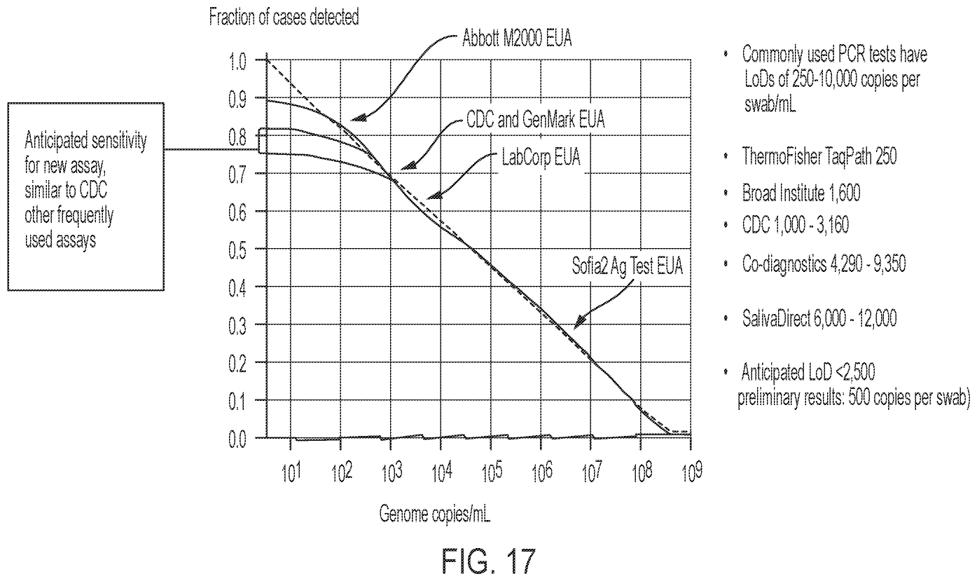

FIG. 17 demonstrates the efficacy and sensitivity of the assay as described herein. Commonly used tests for COVID-19 have limits of detection (LoD) of about 250-10,000 copies per swab per milliliter (mL). As can be seen from the graph of FIG. 15, the LoD of the present COVID-19 detection assay is less than 2500 and in practice is as low as 500 copies per swab.

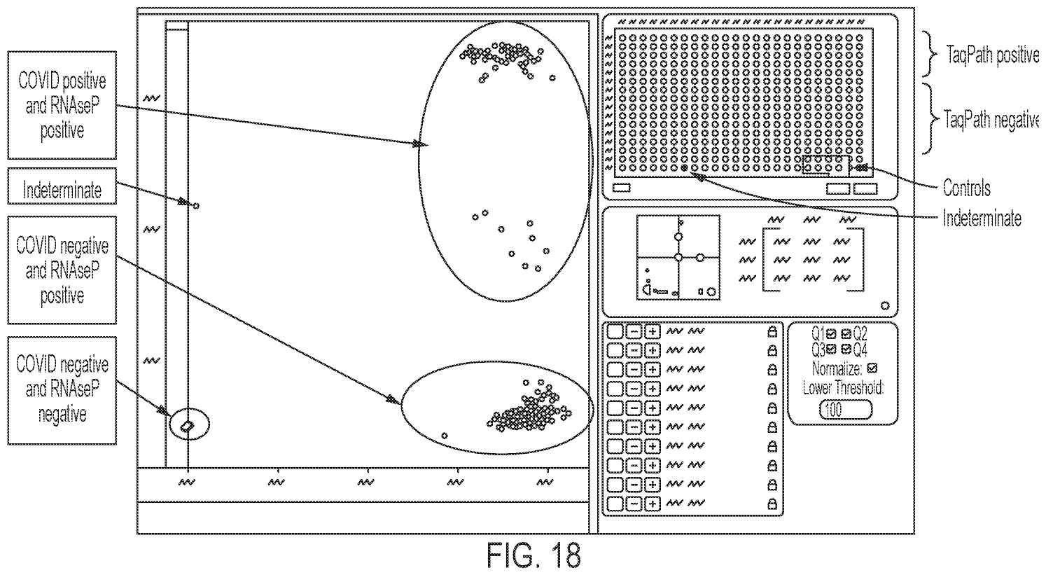

In one study, using the present workflow, approximately 300 symptomatic patients in the clinic with upper respiratory symptoms and fever were dual sampled with dry anterior nares swabs. The samples were first interrogated by TaqPath. Among these, the duplicate swab samples from patients yielding the first 40 positive tests and the first 100 negative tests were analyzed by the assay as presently described. FIG. 18 presents the raw data from this trial, demonstrating that of the 40 positive samples, 39 of the second samples had 2 of 2 positive test results in the duplicate sample, and of the 100 negative samples, 99 had 0 or 1 test positive. This is consistent with virtually perfect assay performance, and 138 of 140 tests in concordance with TaqPath exceeds the 95% accuracy standard set by the Food and Drug Administration.

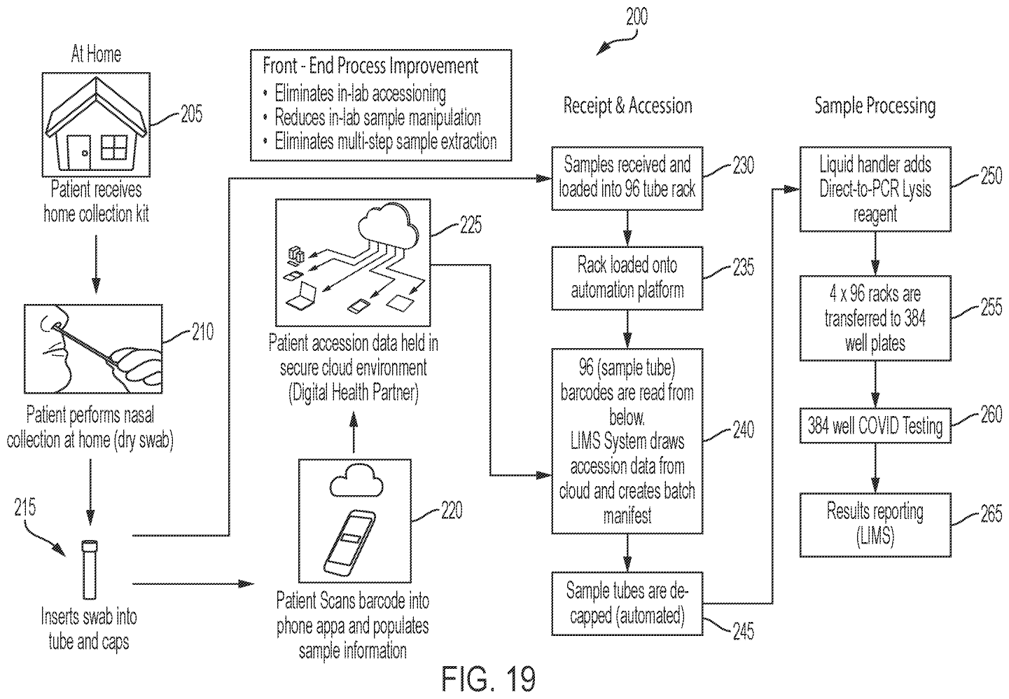

FIG. 19 illustrates method steps of an exemplary process 200 for receiving a sample from a patient and processing the sample. In a first step 205, a collection kit including a device 10 is provided to a patient, possibly at the patient's home. The patient (or a caregiver for the patient) then performs self-collection in step 210, including a step of removing collection assembly 50 from tube 20, as described previously. The collection step 210 may be a nasal collection using a swab 68 of the device 10. After collecting the sample as instructed, the patient inserts the collection assembly 50 into the tube 20 to return the sample-containing device 10 to the engaged configuration 12, optionally rotating the collection assembly 50 to screw it into the tube 20. The patient may then scan a barcode of the device 10 in step 220 to associate his or her identity with the sample. The patient may scan the barcode using an app on a smartphone. This in turn, in step 225, sends the patient accession data to a secure cloud environment.

The patient then returns the device 10, in some cases via delivery service, and it is received by the processing facility in step 230, and loaded into an analytical apparatus 80. Multiple devices 10 from numerous patients may be collected. These devices 10 may engage with a sample rack 82; for instance, the devices 10 may be placed in the wells of a 96-well plate. As described, the devices 10 may be held in place by the projections 27 of tubes 20.

In step 235, the rack of samples may be loaded onto an automation platform, and in step 240 the barcodes of the devices 10 are read. The barcodes may be placed on the bottoms of tubes 20 (see FIG. 2E) so that they can be read from below by a scanner 84 of the analytical apparatus 80. The LIMS system to which the scanner 84 is connected can then draw accession data from the cloud and create a batch manifest for the analytical run.

In step 245, the devices 10 are decapped, in some aspects by an automated decapping machine, such that their secondary caps 40 are removed and the devices 10 are in disengaged configuration 18. Next, in step 250, a liquid handler can then add a processing reagent, such as a direct-to-PCR lysis reagent, to the sample via the primary cap 30 in a contactless fashion. During this step, the samples may be heated, agitated, or further treated. The incubation time for lysis, PCR, and so forth, will be determined by the operator depending on the sample type.

Optionally, in step 255, liquid containing the sample can be moved from the tubes 20 of the devices 10 of four different 96-well plate racks 82, and a smaller volume can be transferred to a 384-well plate, so that more samples can be processed in a single run. In step 260, the sample is tested. For example, when the samples are derived from patients suspected of having COVID-19, an RT-PCR workflow may be employed. In other instances, and for other sample types, the analysis may start with a lytic step, or with a step designed to reduce impurities and interference with signal generated. In some instances, endpoint PCR may be used to generate a readout. The results are then interpreted in step 265 and reported to LIMS, where they are stored for later access by the patient and/or medical personnel.

Although reference has been made herein to testing of patient samples for SARS-Cov-2 and COVID-19, it will be appreciated that the device 10 of the present disclosure can be employed for numerous sample types and for many types of analyses. The device 10 may be used to test for the presence of numerous pathogens and to assist with diagnosis of numerous conditions, including but not limited to human papilloma virus (HPV), respiratory syncytial virus (RSV), influenza viruses, tuberculosis (TB), and others.

Alternatively, the sample obtained using the device 10 may be from an animal, such as an insect or an agricultural mammal. In one aspect, the sample may be obtained from a foodstuff in the food supply change. The sample may instead be obtained from a plant or a portion of a plant, such as a seed, a leaf, a stalk, a spore, a cotyledon, a root, or the like. The sample may be collected from soil or environmental water sources. The device 10 may be used in an agricultural application, or for human disease diagnostics, or a veterinary application. The sample buffer added by the liquid handler, and the conditions of the downstream analysis, can be modified as necessary by the sample type.

In one aspect, an information management system may be integrated with equipment, and the devices 10 including samples may be bar-coded for individual identification. Each device 10 may be coded in multiple ways; for example with a one-dimensional code which is readable by a human, as well as a two-dimensional bar code for machine reading. When read by a machine, each sample can be traced and recorded, with analytical system interacting with software or an application, resulting in the analytical data being stored locally or in cloud-based storage.

### EXAMPLES

EXAMPLE 1: Polyester swabs stored dry demonstrate equivalent performance to foam swabs for detection of low and moderate SARS-CoV-2 viral loads. Paired human clinical matrix swabs were collected from volunteers. Each individual was given a US Cotton #3 polyester swab and a US Cotton #3 ARS swab. The swabs were self-collected and prepared in the following order: 1. Left nostril—US Cotton #3 (5 revolutions); 2. Right nostril—US Cotton #3ARS (5 revolutions); 3. Left nostril—US Cotton #3ARS (5 revolutions, same swab as #2); 4. Right nostril—US Cotton #3 (5 revolutions, same swab as #1). The swabs were collected within a four-hour window followed by inoculation with SARS-CoV-2 Positive Matrix and experimental testing. The SARS-CoV-2 positive pool was produced by combining previously confirmed high-positive patient samples in VTM and stored at 4° C.

The positive pool was added directly to swabs through a procedure that mimicked a nasal swabbing action. Each swab was submerged into a single 1.5 mL microcentrifuge tube containing 40 μL of the SARS-CoV-2 positive pool mixture and “abraded/circled” five times in a single direction against the tube wall, followed by five times in the opposite direction while the viral solution absorbed into the swab. The inoculated swab was returned to the 15 mL conical tube with a screw cap.

The RNase P delta Ct value for the paired means was 0.3, indicating that the swabs are equivalent in terms of sample collection capabilities.

US Cotton #3ARS polyester swab performs similarly to the US Cotton #3 polyester swab in the detection of SARS-CoV-2 by RT-qPCR and RNase P by qPCR. The RT-qPCR detection of low-positive SARS-CoV-2 clinical samples was similar between the US Cotton #3ARS and US Cotton #3 polyester swabs as well as the Copan polyester swabs. Clinical patient samples collected using the US Cotton #3ARS polyester swabs were stable for the detection of SARS-CoV-2 and RNase P at elevated temperatures (40° C.-32° C.) for 48+8 hours. In summary, the US Cotton #3ARS polyester swab performs well with the device of the present disclosure, as does the US Cotton #3 and Copan polyester swabs.

EXAMPLE 2: A dry swab elution process that can be used for standard extraction and qPCR testing. Direct lysis may be conducted via the following direct lysis protocol: first, fresh 1× tris(2-carboxyethyl)phosphine (TCEP) lysis buffer may be made optionally from a 100× stock (100× TCEP is stable for up to 30 days at room temperature.) 1×TCEP lysis buffer is 2.5 mM TCEP, 1 mM ethylenediaminetetraacetic acid (EDTA), pH 7.0-8.0. The steps of the method may include eluting a dry swab with a volume of 1× TCEP (such as about 400 microliters); vortexing for about 30 seconds with intermittent pulsing; heating at about 90 degrees Celsius for about 10 minutes; removing from heat and passively cooling the samples to room temperatures; spinning down samples to remove condensation from the sample tube lid; and proceeding to PCR This protocol is compatible with US Cotton v3, 3ARS (polyester), and 1536 swabs in a 1.5 mL tube. The swab bud should be submerged in TCEP during the heating step.

In one aspect, a negative extraction control (NEC) generates a Ct less than or equal to 37 for none of three COVID-19 targeting assays, the RT-qPCR run passes. Otherwise, the user should repeat the run. If an MS2 spike-in control for the NEC produces a Ct less than or equal to 33, the RT-qPCR run passes. If a COVID-19 RNA control generates a positive signal, the RT-qPCR run passes.

EXAMPLE 3: Sars-CoV-2 nasal specimens are stable when exposed to uncontrolled temperature transport conditions. Mimicking warm- and cold-climate shipment, surrogate specimens were stable following either 72 hours of a high-temperature excursion or two freeze-thaw cycles. Testing included the use of “variable” human matrix and dry transport conditions to determine the stability and RT-PCR detection of SARS-CoV-2 under “winter” transport conditions. The effect of freeze-thaw conditions on the US Cotton 1536 Polyester Swab was interrogated. Paired swabs were collected from human volunteers followed by spiking pooled SARS-CoV-2 clinical matrix, 10× LoD (approximately 2500 GCEs/swab). In addition to SARS-CoV-2 testing, overall human sample collection and stability was assessed by RNase P testing.

The US Cotton 1536 and 1536-CR polyester swabs perform similarly to the Copan polyester swab in the detection of SARS-CoV-2 by RT-qPCR and RNase P by qPCR. The detection of low-positive SARS-CoV-2 clinical matrix by RT-qPCR was comparable between Copan, US Cotton 1536 and 1536-CR polyester swabs. The US Cotton 1536 and Copan polyester swabs are analogous in the detection of SARS-CoV-2 and RNase P when exposed to elevated temperatures (32° C.-40° C.) for 48+8 hours. Moreover, US Cotton 1536 polyester swabs stored at 4° C. or exposed to two freeze-thaw cycles are indistinguishable in the detection of SARS-CoV-2 by RT-qPCR and RNase P by qPCR. In summary, the US Cotton 1536 and 1536-CR polyester swabs perform well, as does Copan polyester swab, for the detection of SARS-CoV-2 by RT-qPCR and RNase P by qPCR.

Therefore the device of the present disclosure enables patient samples to be transported at ambient temperature, or environmental temperature, or variable temperatures, with or without refrigeration, to produce reliable and reproducible results.

EXAMPLE 4: self-collected patient specimens yield sufficient material for molecular testing, as demonstrated by RNase P detection. Testing included the use of “variable” human matrix and dry transport conditions to determine the stability and RT-PCR detection of SARS-CoV-2. The goal of this experiment was to directly compare US Cotton Polyester Swab #3 (version 1) to the Copan Polyester Swab. Paired swabs were collected from human volunteers followed by spiking “hot” human clinical matrix, 10× LoD. In addition to SARS-CoV-2 testing, overall human sample collection and stability was assessed by RNase P qPCR testing, and self-collected, paired swabs from SARS-COV-2 positive patients. The US Cotton Polyester Swab #3 performs similarly to the Copan polyester swab in the stability and RT-qPCR detection of SARS-CoV-2 under dry transport conditions using variable human matrix. There was no effect of temperature under dry transport conditions using variable human matrix for the detection of SARS-CoV-2 from US Cotton Polyester #3 or Copan polyester swabs. Swabs were stable at elevated temperatures for 48+8 hours and at 4° C. for 72+8 hours. The process of self-collection results in a robust sample that has sufficient material for testing as demonstrated by RNase P detection. The dry swab elution procedure produces enough material for RNase P and SARS-CoV-2 detection.

EXAMPLE 5: Investigation of swabs for use as sampling members in the device. We assessed multiple swab characteristics including the swab material, stem, swab width and length. These were all tested with the goal of creating a device for ultra-high throughput testing. We performed numerous studies validating nasal specimen collection stability using a polyester spun swab and then ultimately tested numerous swab types that could be manufactured at high capacity for the creation of a self-collection device.

Devices and methods related to collections and analysis of biological samples are described herein. While a limited number of aspects have been described, those skilled in the art, having benefit of the above description, will appreciate that other aspects may be devised which do not depart from the scope of the present disclosure. In addition, it should be noted that the language used in the specification has been principally selected for readability and instructional purposes, and may not have been selected to delineate or circumscribe the subject matter. Accordingly, the present disclosure is intended to be illustrative, but not limiting, of the scope of its contents.

1-21. (canceled)

22. A device for collecting a biological sample, comprising:
a tube defining a tube body and extending from an open end to a closed end, the tube body defining an exterior surface, an interior surface, and a tube volume bounded by the interior surface; and
a collection assembly at least partially engageable with the open end of the tube to define an engaged configuration, the collection assembly comprising:
a primary cap defining a cap body and extending from a first end to a second end, the cap body defining an outer surface, the cap body comprising:
a first portion including the first end and defining a receiving portion,
a second portion including the second end, the second portion defining a cavity open to the second end, and
an opening in the primary cap,
a sampling member comprising a shaft extending from a connecting end to a sampling end; and
a secondary cap comprising a sealing portion, the sealing portion sized to fit in the cavity of the primary cap,
wherein the connecting end of the sampling member is coupled to the primary cap at the receiving portion;
wherein in the engaged configuration, the opening of the primary cap is open to the tube volume.

23. The device of claim 22, further comprising a sampling member comprising a shaft extending from a connecting end to a sampling end, the connecting end being coupled to the primary cap at the receiving portion.

24. The device of claim 22, wherein the outer surface of the primary cap engages the tube.

25. The device of claim 22, wherein the outer surface of the primary cap defines a thread.

26. The device of claim 25, wherein the secondary cap comprises a proximal portion and a distal portion, the proximal portion containing the sealing portion and defining a thread on an external surface, the sealing portion sealing the opening in the primary cap.

27. The device of claim 26, wherein the thread of primary cap is formed with a first handedness, and the thread of the second cap is formed with a second handedness opposite the thread of the primary cap.

28. The device of claim 22, wherein the receiving portion of the primary cap comprises a thread for engaging the the secondary cap.

29. The device of claim 22, wherein the sampling member comprises a swab fixed to the sampling end.

30. The device of claim 22, wherein the closed end of the tube defines at least one projection on a portion of the exterior surface, the projection being capable of engagement with a component of an analytical apparatus.

31. The device of claim 22, wherein the opening in the primary cap fluidically connects the interior of the tube with a fluid source external to the primary cap when the secondary cap is removed.

32. The device of claim 22, wherein the primary cap further comprises a second opening for air evacuation when the secondary cap is removed.

33. The device of claim 22, wherein the shaft is hollow.

34. The device of claim 22, wherein the shaft is solid.

35. The device of claim 22, wherein the secondary cap is removably connected to and resealable with the primary cap.

36. The device of claim 22, wherein the tube comprises a raised shoulder for interfacing with the analytical apparatus.

37. A system comprising the device of claim 22, and an analytical apparatus.

38. A method of assaying a sample, the method comprising:
disengaging the collection assembly of the device of claim 22 from the tube;
collecting a sample from the patient with the sampling member;
engaging the collection assembly with the tube to define a processing configuration so that the sample is sealed in the device;
placing the device in the processing configuration in an analytical apparatus such that the closed end of the tube engages a portion of the analytical apparatus;
removing the secondary cap of the device; and
analyzing the sample.

39-66. (canceled)
